(19) United States
(12) Patent Application Publication
(10) Pub. No.: US 2026/0358248 A1
(43) Pub. Date: Nov. 12, 2026

(54) DEVICE FOR DETECTING RADIATION BACKSCATTERED BY AN OBJECT

(71) Applicant: COMMISSARIAT A L'ÉNERGIE ATOMIQUE ET AUX ENERGIES ALTERNATIVES, Paris (FR)

(72) Inventors: Luc ANDRE, Grenoble cedex 09 (FR); Guillaume BLANQUER, Grenoble cedex 09 (FR); Martin LEJOSNE, Grenoble cedex 09 (FR)

(73) Assignee: COMMISSARIAT A L'ÉNERGIE ATOMIQUE ET AUX ENERGIES ALTERNATIVES, Paris (FR)

(21) Appl. No.: 19/673,258

(22) Filed: May 11, 2026

(30) Foreign Application Priority Data
May 12, 2025 (FR) FR2505008

(51) Int. Cl. G01N 21/47; G01N 33/483

(52) U.S. Cl. CPC G01N 21/474; G01N 33/4833; G01N 2021/4709; G01N 2201/0668; G01N 2201/068

(57) ABSTRACT

Device for measuring radiation (12, 12₁, 12₂, 12₃, 12₄) backscattered by an object (2), comprising:
at least one light source (10) configured to emit a light beam (11) towards a surface(S) of the object, forming an illumination zone;
a photodetector (22, 22₁, 22₂, 22₃, 22₄) for detecting the radiation backscattered by the object on a detection surface;
   the device comprising:
a radiation collector (20, 20₁, 20₂, 20₃, 20₄), extending along a collection surface describing at least one annular sector and larger than the detection surface;
the collection surface being arranged facing or against the surface of the object,
such that the collector is designed to return the backscattered radiation (12, 12₁, 12₂, 12₃, 12₄) incident on the collection surface to the photodetector.

### TECHNICAL FIELD

The technical field of the invention is that of measuring radiation backscattered by an object, for example living body tissue.

### PRIOR ART

Diffuse reflectance spectroscopy, often referred to by the acronym DRS, consists in exploiting light backscattered by a scattering object subjected to illumination, generally point illumination. This technique proves to be effective for characterizing optical properties of objects, in particular scattering or absorption properties.

Employed on the skin, this technique makes it possible for example to characterize the skin, as described in document EP2762064. The authors of that document describe a measuring probe intended to be applied against the skin. This probe comprises a central optical fibre, referred to as excitation fibre, intended to direct a light beam towards a skin object. Optical fibres, arranged around the central fibre and referred to as detection fibres, collect an optical signal backscattered by the skin. Means for carrying out spectral analysis of the optical signal thus collected, coupled to computing algorithms, make it possible to estimate parameters of the dermis, in particular the concentration of certain chromophores, for example oxyhaemoglobin or deoxyhaemoglobin, but also parameters governing the travel of photons through the dermis, in particular the reduced scattering coefficient μs' and the absorption coefficient Ha. The applications are numerous, and may for example make it possible to detect and quantify concentrations of elements in the skin, or to access information relating to the oximetry of blood vessels passing through the skin, or else respiratory parameters, such as heart rate. Some applications are described in EP2762064, and also in the publication Haahr R., “An Electronic Patch for Wearable Health Monitoring by Reflectance Pulse Oxymetry”, IEEE Transactions on biomedical circuits and systems, 2011, or else in patents EP4394360, EP3311138, EP3236241, EP3210531, EP3338632, or else WO2022076381.

In accordance with the principles of light scattering in a scattering medium, the mean depth of penetration of photons into a scattering object increases with the spacing between the excitation fibre and the detection fibre. In other words, the more the distance between the excitation fibre and the detection fibre increases, the more the result is representative of the optical properties of the deep layers of the medium under examination. By contrast, the more this distance decreases, the more the result is representative of the surface layers of the scattering medium.

Document EP3054281 describes a device comprising an optical system between optical fibres and an object to be characterized, for example the skin of an individual. In that device, the optical fibres are arranged at a distance from the object to be characterized. An optical system with a magnification greater than 1 is arranged between the fibres and the object to be characterized. The optical system, arranged at a distance from the characterized object, optically couples the optical fibres with point elementary zones on the surface of the object.

A more compact device has been described in EP350266. That device comprises an image sensor connected to a bundle of optical fibres, said bundle being applied directly against the object. The bundle of optical fibres is configured to transport an image of the backscattered radiation emanating from the surface of the object to the pixel matrix of the image sensor. However, in such a configuration, the distance, parallel to the surface of the object, between the centre of the light source and the pixel matrix cannot be less than 3 or 4 mm.

The inventors are proposing a device more sensitive than those described in the prior art. This involves in particular improving the signal-to-noise ratio of the intensity of the detected backscattered radiation. The resolution of the device, that is to say its ability to detect fine variations in composition of the object under analysis, varies with the square root of the intensity of the detected backscattered radiation. It is therefore important to increase the intensity of the detected backscattered radiation while at the same time minimizing detection noise.

The proposed device is also well suited to a coherent light source by virtue of having lower sensitivity to the formation of diffraction patterns on the surface of the sample resulting from constructive and destructive interference.

### SUMMARY OF THE INVENTION

A first subject of the invention is a device for measuring radiation backscattered by an object, comprising:
at least one light source configured to emit a light beam, in an illumination spectral band, towards a surface delimiting the object, so as to form an illumination zone on said surface;
a photodetector for detecting the radiation backscattered by the object when said object is illuminated by the light source, the photodetector extending along a detection surface;
a radiation collector, extending along a collection surface;
   wherein
the collection surface describes at least one annular sector extending around a centre, the collection surface being larger than the detection surface;
the collection surface is arranged facing or against the surface of the object;
the radiation collector is configured to return radiation incident on the collection surface to the photodetector, the collector being optically coupled to the photodetector;
so that, when the centre around which the collector extends is arranged in the illumination zone, the collector is designed to return the backscattered radiation incident on the collection surface to the photodetector.

According to one possibility, the or each collector is formed of a Fresnel zone plate, the collector being formed of concentric portions alternating between transparent and opaque portions, configured to focus the radiation incident on the collector towards the photodetector to which said collector is optically coupled.

According to one possibility, the or each collector is formed of concentric portions alternating between portions respectively of a first index and of a second index, greater than the first index, and configured to focus the radiation incident on the collector towards the photodetector to which said collector is optically coupled.

According to one possibility, the or each collector is formed of an optical structure of metasurface or two-dimensional photonic crystal type, configured to focus the radiation incident on the collector towards the photodetector to which said collector is optically coupled.

The collector may extend at a distance, referred to as backscatter distance, from the centre C, so as to return the radiation backscattered by the object at said backscatter distance to the photodetector.

According to one possibility, the device comprises:
multiple photodetectors;
multiple concentric collectors, extending around the centre, each collector being arranged at a distance from the centre that is different from the other collectors, each collector being optically coupled to a different photodetector.

Each collector is thus configured to collect radiation backscattered by the object at a backscatter distance that corresponds to the distance between the collector and the centre, that is to say to the radius of the collector.

The collection surface area may be less than 10 mm² or 5 mm² or 1 mm².

Each photodetector may be arranged facing the collector to which it is coupled.

The radial width of the or each collector is less than 500 μm or 250 μm or 100 μm. Radial is understood to mean a radius of the collector passing through its centre.

According to one possibility:
at least one collector is subdivided into multiple elementary collectors extending at one and the same distance from the centre;
each elementary collector is optically coupled to an elementary photodetector. Each elementary photodetector thus forms the photodetector.

Each elementary collector may extend along an angular extent, around the centre, of less than 90° or less than 45° or less than 30° or less than 15°.

According to one possibility, the device comprises a deflector, configured to deflect the light beam reaching the object towards at least one collector.

The illumination spectral band may extend in the visible region or in the infrared.

A second subject of the invention is a method for detecting radiation backscattered by an object, using a device according to the first subject of the invention, the method comprising the following steps:
a) placing the device on the object, such that the centre is located in the illumination zone and the or each collector is arranged against or facing the surface of the object;
b) illuminating the object using the light source, so as to form an illumination zone on the surface of the object,
c) using the or each photodetector to detect the radiation backscattered by the object and returned by the collector optically coupled to said photodetector.

The method may comprise determining an optical scattering and/or absorption property as a function of an intensity of the backscattered radiation, at various backscatter distances.

The method may comprise determining a concentration of a species absorbing light in the illumination spectral band.

The invention will be better understood on reading the disclosure of the exemplary embodiments presented, in the remainder of the description, with reference to the figures listed below.

### FIGURES

FIG. 1A shows one example of a device in a plane XZ perpendicular to the surface of the object.

FIG. 1B shows the example of a device in a plane XY parallel to the surface of the object.

FIG. 1C is a view of one example of a device in a plane XZ perpendicular to the surface of the object.

FIG. 1D is a view of one example of a device in a plane XZ perpendicular to the surface of the object.

FIG. 2A shows a variant of the device from FIG. 1A, comprising a deflector.

FIG. 2B schematically shows the deflector shown in FIG. 2A.

FIG. 3A is a model of the travel of photons through an object.

FIG. 3B shows the angle of backscattered radiation with respect to the direction normal to the surface of the object for two types of light source (collimated or Lambertian), with and without a deflector.

FIG. 4 shows one example of a Fresnel zone plate collector.

FIG. 5A shows the obtaining of a collector as shown in FIG. 4.

FIG. 5B shows the position of a photodetector with respect to a Fresnel zone plate.

FIG. 5C shows the luminous intensity focused on a photodetector by a Fresnel zone plate.

FIG. 5D is an intensity profile along a line of FIG. 5C passing through the centre of the image.

FIG. 6 shows the lengths of the mean trajectories travelled by photons through the object shown in FIG. 1A.

FIG. 7 shows a variant whereby each collector is subdivided into elementary collectors located at one and the same distance from one and the same centre, each elementary collector being optically coupled to one and the same elementary detector.

FIG. 8 shows a processing circuit configured to receive detection signals generated by two photodetectors.

### DESCRIPTION OF PARTICULAR EMBODIMENTS

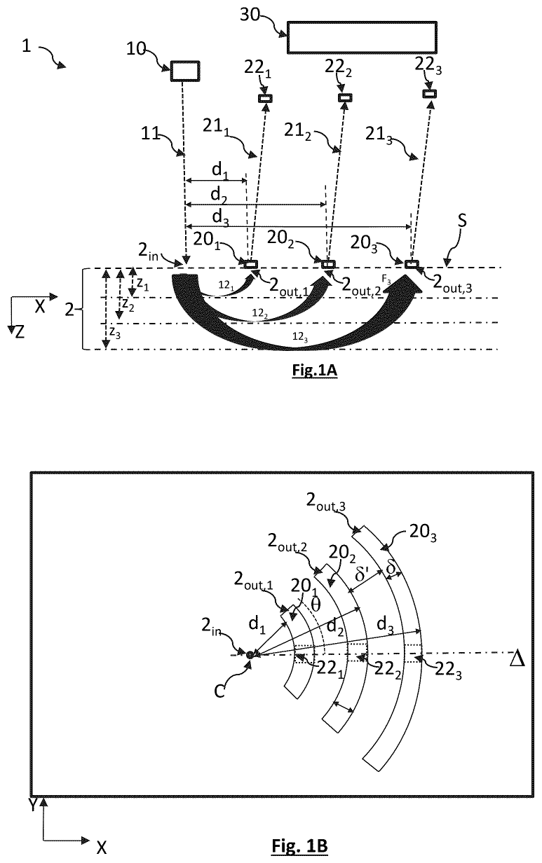

FIGS. 1A to 1D schematically show the main components of a device 1 according to the invention. The device 1 comprises a light source 10 configured to emit an illumination beam 11 towards the surface S of an object 2. The illumination beam is emitted in an illumination spectral band Δλ. Preferably, the illumination spectral band is narrow, and extends over less than 10 nm or less than 5 nm. The light source 10 may be a laser source or an LED coupled to a bandpass filter defining the illumination spectral band. The illumination spectral band may extend in the visible region or in the infrared region.

For example, when seeking to determine a concentration of glucose in the object, the illumination spectral band is in the near infrared. The absorption cross section of tissue is generally low, and glucose has absorption spectral bands, for example at 1540 nm.

The illumination beam 11 forms a luminous trace, referred to as illumination zone 2ᵢₙ, on the surface S of the object. The illumination zone 2ᵢₙ corresponds to an intersection between the illumination beam 11 and the surface S of the object. In the example shown, the illumination zone is considered to be a point, or considered as such, this being the preferred embodiment. It preferably extends along a diameter or longer diagonal of less than 1 mm.

The object 2 may be a body area of a living being, for example skin, so as to characterize the absorption or scattering properties of the skin, or to estimate a physiological parameter, for example an oxygen or glucose content, or a breathing rate, as described in the patents cited in the prior art.

Under the effect of illumination, a portion of the light originating from the illumination beam 11 scatters in the object 2, and is backscattered thereby so as to form backscattered radiation emanating from the surface S of the object. As described in the prior art, the backscattered radiation may be detected, thereby making it possible to determine optical properties of the object, for example optical scattering or absorption properties. It is also or alternatively possible to estimate the concentration of a target species, absorbing light in the illumination spectral band, in the object. Such a target species will be referred to hereinafter as a chromophore. This may be a molecule, for example glucose, or oxygen, or CO₂, the concentration of which in the object under analysis it is desired to determine.

An optical scattering or absorption property is understood to mean an absorption coefficient, or a scattering coefficient, or a reduced scattering coefficient, these variables being known to those skilled in the art and described in application EP3054281.

FIG. 1A schematically shows, using arrows, the travel, through the object 2, of photons scattering in said object so as to form the backscattered radiation. The backscattered radiation emanates from the surface S of the object 2 in various backscatter zones 2_{out,1}, 2_{out,2}, 2_{out,3} distinct from the illumination zone 2ᵢₙ. The term “backscatter distance” denotes a distance d between a backscatter zone and the illumination zone 2ᵢₙ.

FIG. 1A is a view along a plane XZ, where X is a longitudinal axis and Z is a transverse axis, perpendicular to the surface S of the object 2. The figure shows three backscatter zones 2_{out,1}, 2_{out,2}, 2_{out,3} extending respectively at three backscatter distances d₁, d₂ and d₃ from the illumination zone 2ᵢₙ, and from which respective backscattered radiation 12₁, 12₂, 12₃ emanates. The term backscatter zone denotes a zone of the surface of the object the geometry of which is such that the zone may be considered to be located at one and the same backscatter distance from the illumination zone 2ᵢₙ.

FIG. 1B shows a view of the device in a plane XY, where Y is a lateral axis. The plane XY is parallel to the surface S of the object. Each backscatter zone 2_{out,1}, 2_{out,2}, 2_{out,3} describes all or part of a ring, with a small thickness in the plane XY, preferably less than 1 mm, or even 500 μm, or even 100 μm or 50 μm and extending around the illumination zone 2ᵢₙ. The thickness is a radial thickness defined along a radius of the collector passing through the centre C.

As mentioned in EP3054281, the smaller the backscatter distance, the less deep the path, through the object, of the photons forming the scattered radiation extends into the object. Thus, the smaller the backscatter distance, the more representative the backscattered radiation is of a surface portion of the object. By contrast, the greater the backscatter distance, the more representative the backscattered radiation is of a deep portion of the object. In FIG. 1A, the object has been segmented into three adjacent segments:
a surface segment, extending to a depth z₁;
an intermediate segment, extending between the depth z₁ and the depth z₂;
a deep segment, extending between the depth z₂ and the depth z₃.

The backscattered radiation 12₁, emanating from the surface of the object at the backscatter distance d₁, passes through only the surface segment: it is representative of the composition of the object only in the surface segment. The backscattered radiation 12₂, emanating from the surface of the object at the backscatter distance d₂, passes through the surface segment and the intermediate segment: it is representative of the composition of the object only in these two segments. It will be understood that the backscattered radiation 12₃, emanating from the surface of the object at the backscatter distance d₃, is representative of the composition of the object in the three segments.

The intensity of the backscattered radiation at a given backscatter distance is low. The publication Senlik O. et al “Concentric Multipixel Silicon Photodiode Array probes for Spatially resolved diffuse reflectance spectrometry” describes the use of photodiodes forming annular sectors and intended to be applied against the surface of an object. Such a solution presupposes the use of photodiodes with a large surface area, thereby increasing detection noise. This increases the payload signal, but also detection noise. This is not optimum from the point of view of signal-to-noise ratio.

The inventors are proposing a device that makes it possible to increase the collection surface for backscattered radiation while at the same time limiting photodetector detection noise. For this purpose, one important component of the device is an annular collector, describing an annular sector, configured to collect backscattered radiation at one and the same backscatter distance, and to direct the backscattered radiation to a point photodetector. A point photodetector is understood to mean a photodetector with a small surface area, for example less than 10 μm by 10 μm, or even less than 5 μm by 5 μm, ideally of the order of 1 μm by 1 μm.

An annular sector is understood to mean a region present between two concentric circles and two radii delimiting a given angle. It is therefore a “segment” or portion of a circular ring.

The ring or annular sector extends around a centre C. The width of the ring, in the plane XY, is small enough for the ring to be considered to extend at one and the same distance from the light source. The width of the ring is thus preferably less than at least 1/10^{th}, or even 1/20^{th} of the distance separating it from the centre C.

In the example shown, the device comprises three collectors 20₁, 20₂, 20₃ with a width δ=25 μm, and spaced from one another by a spacing δ′ of 100 μm or a few hundred 100 μm. The three collectors 20₁, 20₂, 20₃ are arranged respectively at distances d₁=250 μm, d₂=500 μm, d₃=750 μm from the centre C. When the device is deployed, the centre C is arranged in the illumination zone 2ᵢₙ, in contact with the surface of the object 2.

Each backscatter distance is generally between 100 μm and a few cm, preferably between 100 μm and 1 cm.

In the configuration shown, each collector extends on either side of a median axis Δ, passing through the centre C and forming an axis of symmetry of the collector. Each collector extends, with respect to the median axis, and on either side thereof, along an angle of extent θ that may reach or exceed 45°. The angle of extent θ may reach π (complete ring), but the inventors consider that it is preferable for θ to be less than 90°, or even 60° or 45°. In theory, the higher θ, the larger the collection surface, and the higher the collected intensity of the backscattered radiation. However, trying to achieve an excessively high angle may involve technological constraints. Each collector is configured to focus the backscattered radiation towards a photodetector. The collectors 20₁, 20₂, 20₃ thus respectively focus the backscattered radiation 12₁, 12₂ and 12₃ towards the photodetectors 22₁, 22₂ and 22₃. Downstream of each collector 20₁, 20₂, 20₃, the collected radiation 21₁, 21₂ and 21₃ converges towards the photodetectors 22₁, 22₂ and 22₃, respectively. Each collector is thus optically coupled to a different photodetector than another collector. The coupling between each collector and each photodetector is bijective, in the sense that a collector is optically coupled to a single photodetector and vice versa.

The use of elementary collector/photodetector pairs makes it possible to detect any anomaly, for example when the photodetectors respectively coupled to collectors, with the same surface area and addressing one and the same backscatter distance, detect different intensities. For example, if one of the photodetectors detects a lower intensity than the others, this may be the consequence of a singularity, for example an absorbent point zone or hair. The detected signal considered to be abnormal may then be suppressed.

The signals from photodetectors addressing one and the same backscatter distance may be summed so as to obtain a single intensity for each backscatter distance under consideration.

In the configuration shown in FIG. 1B, the angle of extent θ is the same for the three collectors. According to another possibility, the angles of extent of different collectors are respectably different.

The space between the collectors is opaque and absorbent in the illumination spectral band Δλ, except for at the centre C, so as to allow the illumination radiation 11 to form the illumination zone 2ᵢₙ. FIGS. 1C and 1D show, in the plane XZ, two different possibilities for positioning a photodetector 22 with respect to a collector 20. FIG. 1C shows a configuration in which a photodetector 22 is arranged centred and in line with a collector 20. The collected radiation 21 converges towards the photodetector 22, forming a symmetrical cone. Such a configuration is suitable for the collection of backscattered radiation 12 emanating from the object in a direction normal to the surface S. FIG. 1D shows a configuration whereby the photodetector 22 is arranged off-centre with respect to the configuration shown in FIG. 1C. The photodetector 22 is thus offset with respect to the normal to the collector 20 passing through the centre thereof. Such a configuration is suitable for the collection of backscattered radiation 12 emanating from the object while being inclined, by an angle of inclination α, with respect to the direction normal to the surface S. The collected radiation 21 converges towards the photodetector 22 forming a cone extending about a central axis inclined by an angle α′ with respect to the normal to the detection surface. In FIGS. 1C and 1D, the normal to the surface S is shown in dot-and-dash lines.

In the absence of any deviation of the axis about which the backscattered radiation propagates, through the detection surface, α and α′ are the same.

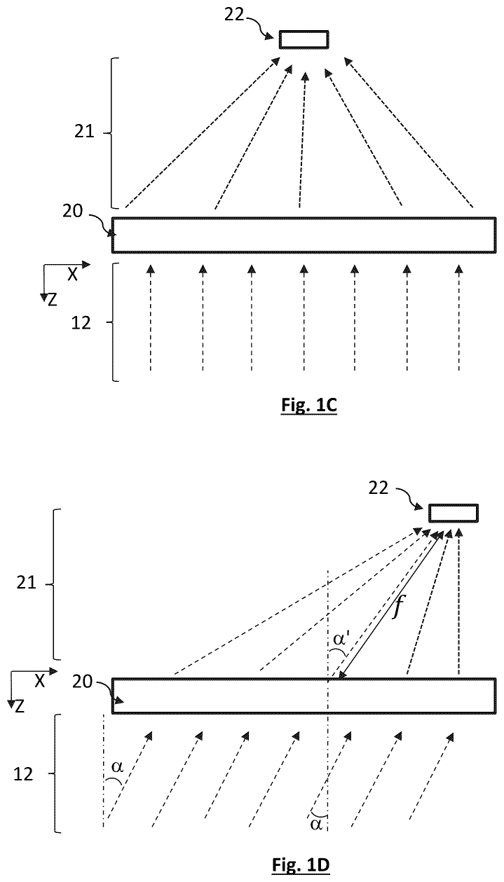

FIGS. 2A and 2B illustrate one variant whereby the device comprises a deflector 13 intended to deflect the illumination beam 11 in the direction of the collectors. The deflector 13 may be formed by a prism, or by an optical microstructure designed to deflect the beam. One example of such a microstructure is shown schematically in FIG. 2B. It comprises pads 13₂, with a thickness of the order of a wavelength, and with a lateral dimension that increases gradually moving away from the centre C towards the one or more collectors. More specifically, the lateral dimension increases along an axis (here the axis X) perpendicular to the propagation axis of the illumination beam 11, between the centre C and the one or more collectors. Along the axis Y, the array is invariant. The pads 13₂ are spaced by a spatial pitch smaller than the illumination wavelength. The pads are formed by a material, preferably a non-metal material, that is transparent to the illumination wavelength and has a refractive index greater than the refractive index of the ambient medium 13₁. The gradual increase in the lateral dimension of the pads 13₂, with a high refractive index, makes it possible to deflect the illumination beam 11, ideally by the order of 45°.

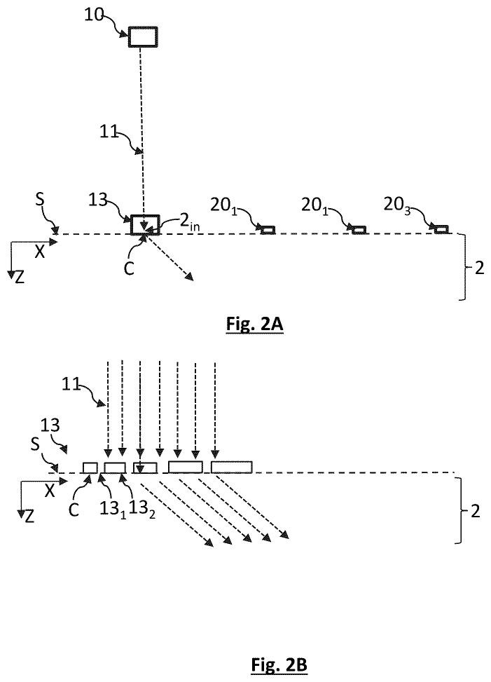

FIG. 3A shows a model of the transportation of photons through a scattering sample formed of milk, taking into account a backscatter distance of 250 μm. It will be recalled that the backscatter distance corresponds to the distance between the illumination beam and the zone of the surface of the object from which the backscattered radiation emanates. Depending on the depth of penetration into the object, the angle of inclination α may vary. The angle of inclination α may be modelled, thereby making it possible to predict the position of each photodetector 22 relative to the collector 20 to which it is coupled. The photodetector is preferably arranged in the optical axis of the collector 20, inclined by the axis α′ with respect to the normal to the surface of the sample. The angle α′ corresponds to the inclination of the axis connecting the centre of the collector to the centre of the photodetector.

The angle α depends on the backscatter distance. FIG. 3B shows the angle α (ordinate axis—unit in degrees) as a function of the backscatter distance (abscissa axis—unit in mm), using various light sources.
curve a: Lambertian light source, propagating along a propagation axis normal to the surface of the sample.
curve b: Lambertian light source, propagating along a propagation axis inclined by 45° with respect to the surface of the sample.
curve c: collimated light source, propagating along a propagation axis normal to the surface of the sample.
curve d: collimated light source, propagating along a propagation axis inclined by 45° with respect to the surface of the sample.

It is observed that, regardless of the type of light source, the more the backscatter distance increases, the closer the angle α of the backscattered radiation with respect to the normal to the surface of the sample gets to 45°. In reality, as may be seen in FIG. 3A, the inclination of the backscattered radiation is distributed around the angle α, said angle corresponding to the angle of inclination for which the backscattered radiation is most intense.

Each photodetector is preferably a point photodetector, or may be considered as such. It may for example be a photodiode. The detection surface of the photodetector is preferably of the order of or less than a few μm² or tens of μm². As an alternative, each photodetector may be formed by one end of an optical fibre, so as to guide light to a remote detector, for example an image sensor.

According to a first possibility, each collector takes the form of a Fresnel zone plate, usually denoted by the acronym FZP. An FZP is formed of concentric portions alternating between transparent or opaque portions in the illumination spectral band Δλ. Each portion is dimensioned so as to form constructive interference at a focal point, thus making it possible to concentrate incident light energy. It will be understood that the focal point corresponds to the photodetector to which the collector is optically coupled. The operating principle of FZPs according to the invention has been described in the publication Moreno V. et al, “High efficiency diffractive lenses: Deduction of kinoform profile”, Am. J. Phys. 65 (6), June 1997.

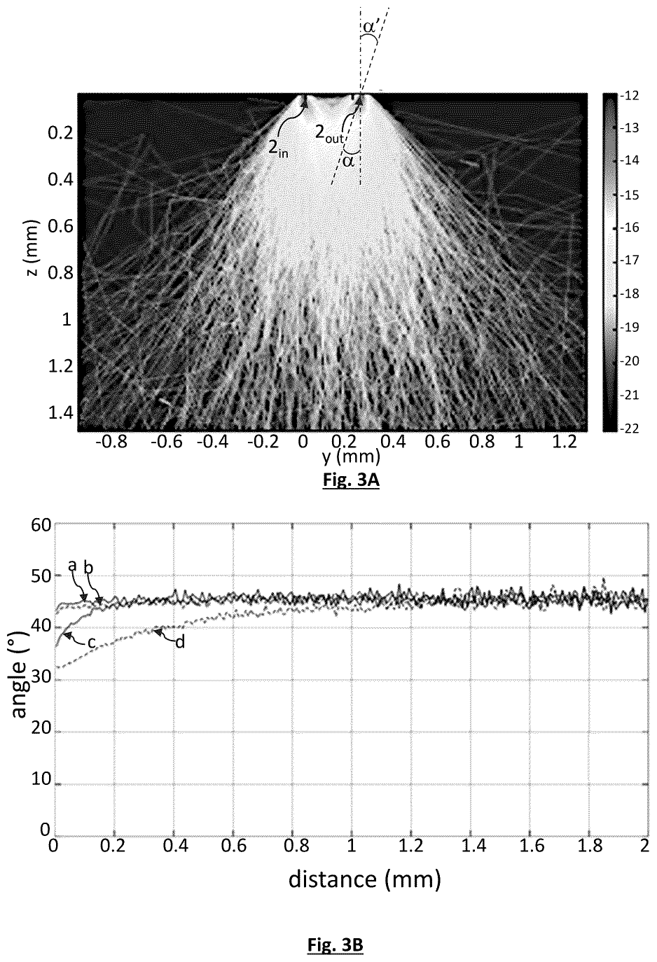

FIG. 4 shows one example of an FZP collector 20, extending along a width of 25 μm and a height of 60 μm. Such a collector may be obtained by a succession of concentric rings assigned a rank m, m being an integer between 1 and M, M corresponding to the ring of maximum radius. Each ring of rank m on either side of a radius Rₘ such that Rₘ²=mλf, where f corresponds to the focal length, that is to say the distance between the photodetector and the collector along the optical axis. λ is the wavelength of the illumination beam.

The materials forming the rings are alternately opaque and transparent in the illumination spectral band Δλ. However, the efficiency of FZP structures is limited due to the presence of opaque portions. Collection efficiency may be improved using portions formed by an alternation of materials having a high index and a low index. From low-index materials, air or silica (refractive index of 1.5) may be chosen. From high-index materials, silica (refractive index of 3.5) or silicon nitride (1.7) may be chosen. The low-index and high-index portions induce a phase shift of π and 2π, respectively. Outside the annular sector 20, a metal deposition makes it possible to obtain opacity. The grayscale level is the luminous intensity. The distribution of the luminous intensity was obtained by modelling using a finite-difference time domain (FDTD) method. The thickness of each high-index ring may be of the order of 1 μm.

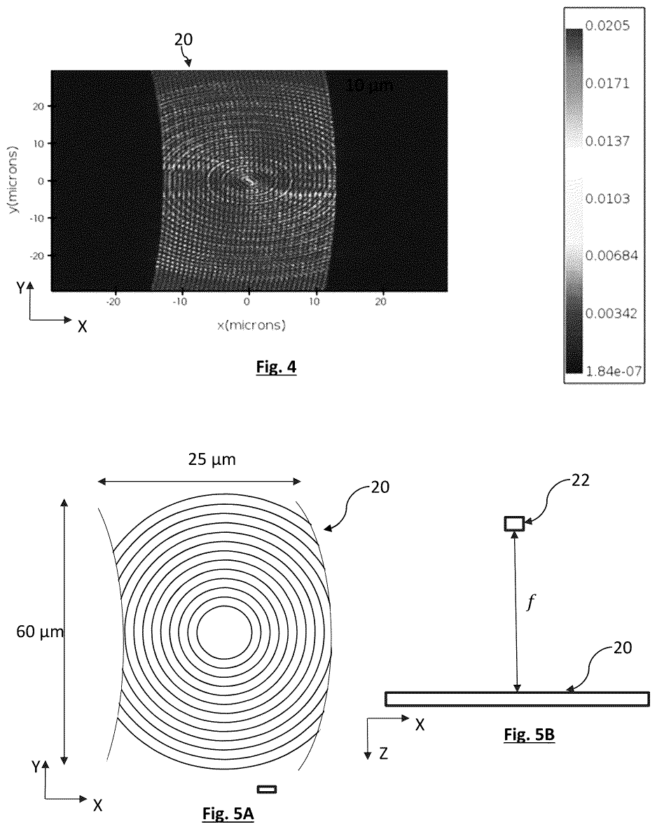

FIG. 5A shows a plan view of the collector, in the plane XY, in which the concentric rings forming the FZP are visible outside the annular sector forming the collector. Realizing the collector comprises forming concentric rings, and then adding a metal deposition outside the annular sector forming the collector.

FIG. 5B is a cross-sectional view, along the plane XZ, of the assembly formed by the collector 20, as shown in FIG. 5A, and a photodetector 22, forming the focal point of the collector 20. The dimension of the photodetector is for example 10 μm×10 μm.

FIG. 5C shows the luminous intensity in a plane XY passing through the photodetector shown schematically in FIG. 5B. The grayscale level corresponds to the luminous intensity. FIG. 5C was obtained by modelling, in the same way as FIG. 3A. It may be seen that the collected light is concentrated on a point zone in the centre of the image. FIG. 5D is an intensity profile along a line passing through the focal point of FIG. 5C. The profile makes it possible to appreciate the concentration of light.

According to another possibility, the collector is formed of photonic crystals or a metasurface. The use of periodic structures that modulate the propagation of light, making it possible to control photon propagation, is well known. Such structures make it possible in particular to focus light. The use of two-dimensional photonic crystals makes it possible to obtain “flat lenses” that focus light towards a precise focal point by exploiting the diffractive and refractive properties of periodic structures.

Regardless of the structure of the collector, in the presence of various collectors, each collector focuses the collected backscattered radiation towards a focal point specific thereto and different from the focal point of the other collectors.

The intensity of backscattered radiation at the distance d, at the wavelength λ may be explained by:

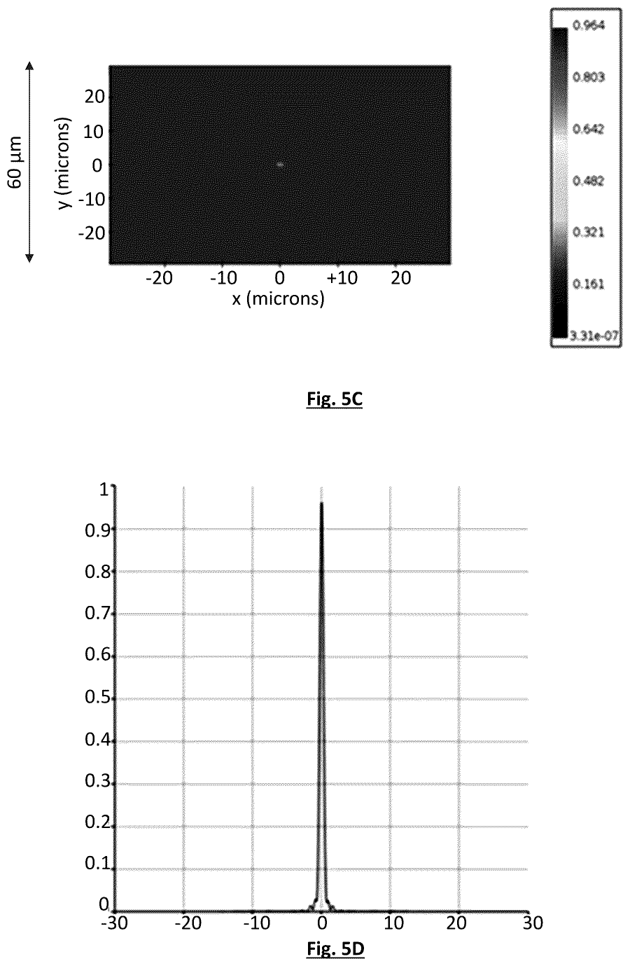

I(λ,d,l)=I₀(λ)·R(λ,d)·e^{-μ}^{ₜ}^{l}e^{-δμ}^{_{c}}^{c}^{_{c}}^{l} (1)

where:
I₀(λ) is the intensity of the illumination beam reaching the illumination zone 2ᵢₙ.
R(λ, d) is the reflectance of the object, without absorption, at the wavelength λ, related to the presence of scatterers in the object, at the backscatter distance d;
e^{-μ}^{ₜ}^{l(d)} is the absorption of the radiation by the object, with the absorption coefficient μₜ, along the trajectory l(d) of the radiation in the object. The trajectory l(d) corresponds to the mean path travelled by the photons emerging from the object at the backscatter distance d, taking into account the curvature represented by the arrows l₁, l₂, l₃ in FIG. 6.
e^{-δμ}^{_{c}}^{c}^{_{c}}^{l(d)} is the variation in the signal caused by the presence of a molecule with a concentration c_{c} and linear absorption δμ_{c} along the trajectory l(d) followed by the radiation.

In the described application, it is sought to determine a modification of the absorption of the medium in the presence of a molecule of interest inducing a variation δμ_{c} in the absorption.

The trajectory l(d) may be considered to be proportional to the backscatter distance d: l(d)=σd (2) where σ is a positive real coefficient. Thus:

I(λ,d)=I₀(λ)·R(λ,d)·e^{-μ}^{ₜ}^{σd}e^{-δμ}^{_{c}}^{c}^{_{c}}^{σd} (3)

The intensity detected by each photodetector is denoted i(λ, d). It depends on a concentration factor K(d) of each concentrator, which corresponds to the ability of a concentrator to concentrate light collected on the photodetector to which it is coupled.

Thus:

i(λ,d)=I₀(λ)·R(λ,d)·K(d)·e^{-μ}^{ₜ}^{σd}e^{-δμ}^{_{c}}^{c}^{_{c}}^{σd} (5)

For each backscatter distance d, it is possible to dimension the collector such that

$$K ( d ) = \frac{{K}_{0}}{R ( λ , d ) {e}^{- {μ}_{t} σ d}} \qquad ( 6 )$$

The variables R(λ, d) and e^{-μ}^{ₜ}^{σd} may be estimated by modelling, or by measurements carried out on phantoms mimicking the object, in the absence of chromophore.

K₀ is defined for each collector as a function of the maximum extent of the first ring (m=1), the radius of which depends on the focal length and wavelength. K₀ may be estimated by modelling and/or by experimental tests.

Taking (6) into account, it is possible to have multiple collectors at various backscatter distances d and the dimension, in particular angular extent θ, of which makes it possible to satisfy Equation (6).

By combining (5) and (6), the luminous intensity detected by each photodetector thus becomes:

i(λ,d)=I₀(λ)·K₀e^{-δμ}^{_{c}}^{c}^{_{c}}^{σd} (7)

The advantage of having two backscatter distances is that of being able, at one and the same time, to implement a ratio:

$$\frac{i ( λ , {d}_{1} )}{i ( λ , {d}_{2} )} = {e}^{- {δμ}_{c} {c}_{c} σ ( {d}_{1} - {d}_{2} )} \qquad ( 8 )$$

This makes it possible to do away with the need for knowledge about the illumination beam and any variations thereof.

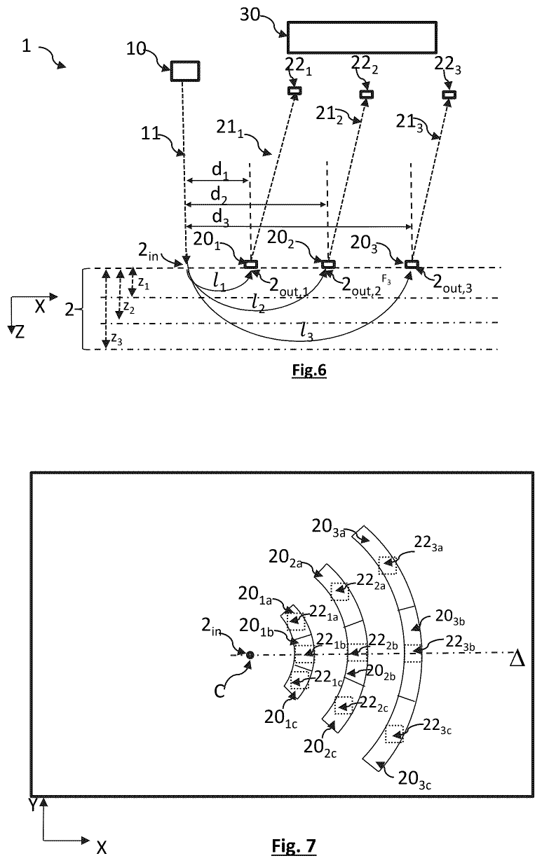

FIG. 7 illustrates one variant whereby each collector is segmented into various elementary collectors. Each elementary collector forming a collector is located at one and the same distance from the centre C. FIG. 7 shows:
The first collector 20₁, formed by three first adjacent elementary collectors 20₁ₐ, 20_{1b}, 20_{1c};
The second collector 20₂, formed by three second adjacent elementary collectors 20₂ₐ, 20_{2b}, 20_{2c};
The third collector 20₃, formed by three third adjacent elementary collectors 20₃ₐ, 20_{3b}, 20_{3c}.

Each elementary collector is optically coupled to an elementary photodetector in a bijective link. Thus:
The first photodetector 22₁ is formed by first elementary photodetectors 22₁ₐ, 22_{1b} and 22_{1c} respectively optically coupled to the first elementary collectors 20₁ₐ, 20_{1b}, 20_{1c}.
The second photodetector 22₂ is formed by second elementary photodetectors 22₂ₐ, 22_{2b} and 22_{2c} respectively optically coupled to the second elementary collectors 20₂ₐ, 20_{2b}, 20_{2c}.
The third photodetector 22₃ is formed by third elementary photodetectors 22₃ₐ, 22_{3b} and 22_{3c} respectively optically coupled to the third elementary collectors 20₃ₐ, 20_{3b}, 20_{3c}.

The elementary photodetectors of one and the same photodetector are connected to one another such that the intensity detected by each elementary photodetector is summed. This makes it possible to add together the intensities respectively detected by the elementary photodetectors addressing one and the same backscatter distance.

The subdivision of a photodetector into multiple elementary photodetectors makes it possible to overcome technological constraints related to the formation of a collector with an excessively high angular extent. It is thus possible to increase the collection surface, and therefore the intensity of the detected backscattered radiation, by employing elementary collectors with a simple design. The angular extent of each elementary collector may be less than 45°, or even less than 30°, or even less than 15° or 10°.

The photodetectors are connected to a processing unit 30, configured to process the detection signals resulting from each photodetector. The processing unit may comprise analogue or digital modules for processing the detection signals, along with a microprocessor for applying an analysis algorithm to the processed detection signals, for example to determine a concentration of a chromophore, or another physiological parameter expressed by the detection signals.

According to one possibility, the analogue module comprises logarithmic amplifiers that convert an input signal into an output voltage proportional to the amplitude of the input signal. Each logarithmic amplifier is configured to be supplied with the detection signal from a photodetector.

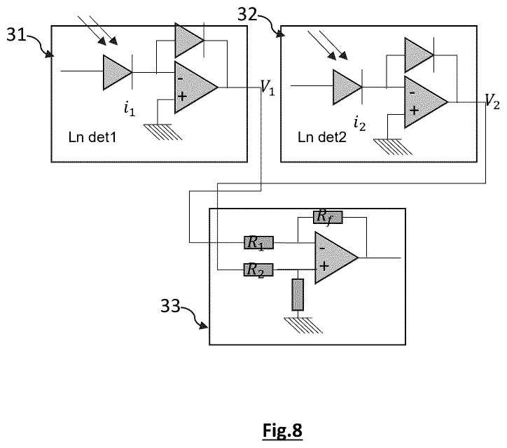

FIG. 8 shows:
a first logarithmic amplifier 31, supplied with the detection signal i₁ corresponding to the first backscatter distance d₁. The output voltage is:

V₁∝ ln(i₁) (9);
a second logarithmic amplifier 32, supplied with the detection signal i₂ corresponding to the second backscatter distance d₂. The output voltage is:

V₂∝ ln(i₂) (9′);
a subtractor 33, supplied by the first and second logarithmic amplifiers, the output voltage of which is:

$${V}_{s} = \frac{{R}_{f}}{{R}_{1}} ( {V}_{2} - {V}_{1} ) . \qquad ( 10 )$$

Combining (9), (9′) and (10) gives:

$${V}_{s} = \frac{{R}_{f}}{{R}_{1}} {δμ}_{c} {c}_{c} σ ( {d}_{2} - {d}_{1} ) \qquad ( 11 )$$

In FIG. 8, it is assumed that R₂=R₁.

Measuring Vₛ thus makes it possible to obtain the sought chromophore concentration c_{c}. The setup shown in FIG. 8 makes it possible to isolate the payload signal, in this case Vₛ, before processing it, for example amplifying it.

The use of concentrators makes it possible to increase the collection surface while at the same time using a small photodetector, thereby making it possible to increase the detected signal while at the same time maintaining a low noise level. Indeed, the dark current of a photodetector may be considered to be proportional to its surface area. The signal-to-noise ratio is increased compared to the configurations from the prior art. This makes it possible to improve the sensitivity of the device when estimating a concentration of a chromophore. This also makes it possible to determine the absorption or scattering properties more precisely.

Moreover, when the light source is coherent, constructive or destructive interference may result in the occurrence of diffraction patterns, or speckle, affecting the spatial uniformity of the backscattered radiation at a given backscatter distance. The use of a collector makes it possible to increase the collection surface and to smooth any spatial nonuniformity of the backscattered radiation.

Each collector is preferably delimited by at least one planar surface. It may be arranged in contact with the sample, or on a layer of a spacer material used to adapt the optical index.

The invention may be implemented on biological tissue, for the applications described in the prior art. It may also be implemented with a view to analysing samples, for example in the cosmetic or agri-food industry.

1. A device for measuring radiation backscattered by an object, comprising:
at least one light source configured to emit a light beam, in an illumination spectral band, towards a surface delimiting the object, so as to form an illumination zone on said surface;
a photodetector for detecting the radiation backscattered by the object when said object is illuminated by the light source, the photodetector extending along a detection surface;
a radiation collector, extending along a collection surface, the collection surface describing at least one annular sector extending around a centre, the collection surface being larger than the detection surface;
wherein
the collection surface is arranged facing or against the surface of the object;
the radiation collector is configured to focus radiation incident on the collection surface to the photodetector, the radiation collector being optically coupled to the photodetector;
so that, when the centre is arranged in the illumination zone, the radiation collector is configured to return the backscattered radiation, incident on the collection surface, to the photodetector.

2. The device according to claim 1, wherein the radiation collector is formed of a Fresnel zone plate, the radiation collector being formed of concentric portions alternating between transparent and opaque portions, configured to focus the radiation incident on the radiation collector towards the photodetector to which the radiation collector is optically coupled.

3. The device according to claim 1, wherein the radiation collector is formed of concentric portions alternating between portions respectively of a first index and of a second index, greater than the first index, and configured to focus the radiation incident on the radiation collector towards the photodetector to which the radiation collector is optically coupled.

4. The device according to claim 1, wherein the radiation collector is formed of an optical structure of metasurface or two-dimensional photonic crystal type, configured to focus the radiation incident on the radiation collector towards the photodetector to which the radiation collector is optically coupled.

5. The device according to claim 1, wherein the radiation collector extends at a backscatter distance, from the centre, so as to focus the radiation, backscattered by the object at said backscatter distance, to the photodetector.

6. The device according to claim 1, comprising:
multiple photodetectors;
multiple concentric radiation collectors, extending around the centre, each radiation collector being arranged at a distance from the centre that is different from the other radiation collectors, wherein each radiation collector is optically coupled to a different photodetector.

7. The device according to claim 1, wherein the collection surface area is less than 10 mm² or 5 mm² or 1 mm².

8. The device according to claim 1, wherein the photodetector is arranged facing the radiation collector to which it is coupled.

9. The device according to claim 1, wherein the radial width of the angular sector is less than 500 μm or 250 μm or 100 μm.

10. The device according to claim 1, wherein:
the radiation collector is subdivided into multiple elementary collectors extending at one and the same distance from the centre;
each elementary collector is optically coupled to an elementary photodetector, so that each elementary photodetector forms the photodetector.

11. The device according to claim 10, wherein each elementary collector extends along an angular extent, around the centre, of less than 90° or less than 45° or less than 30° or less than 15°.

12. The device according to claim 1, comprising a deflector, configured to deflect the light beam reaching the object towards at least one radiation collector.

13. The device according to claim 1, wherein the illumination spectral band extends in the visible region or in the infrared.

14. A Method for detecting radiation backscattered by an object, using the device according to claim 1, the method comprising:
a) placing the device on the object, such that the centre is located in the illumination zone and the radiation collector is arranged against or facing the surface of the object;
b) illuminating the object using the light source, so as to form an illumination zone on the surface of the object,
c) using the photodetector to detect the radiation backscattered by the object and focused by the radiation collector optically coupled to said photodetector.

15. The method of claim 14, wherein the method further comprises determining an optical scattering and/or absorption property as a function of an intensity of the backscattered radiation.

16. The method of claim 14, wherein the method comprises determining a concentration of a species absorbing light in the illumination spectral band.
